(19) United States
(12) Patent Application Publication
(10) Pub. No.: US 2025/0283796 A1
(43) Pub. Date: Sep. 11, 2025

(54) METHOD FOR ANALYZING THE LEAF GROUP COMPOSITION OF A CIGARETTE SAMPLE BASED ON THERMAL CHARACTERISTICS OF TOBACCO LEAVES

(71) Applicant: CHINA TOBACCO YUNNAN INDUSTRIAL CO., LTD., Kunming (CN)

(72) Inventors: Ji YANG, Kunming (CN); Qianxu YANG, Kunming (CN); Wei ZHE, Kunming (CN); Hongtao FENG, Kunming (CN); Chunbo LIU, Kunming (CN); Shiyun TANG, Kunming (CN); Zhenjie LI, Kunming (CN); Jianming CHEN, Kunming (CN); Guofu ZHOU, Kunming (CN); Wei JIANG, Kunming (CN); Xiaoxi SI, Kunming (CN); Yong LI, Kunming (CN); Fengmei ZHANG, Kunming (CN); Zhouhai ZHU, Kunming (CN)

(21) Appl. No.: 18/791,784

(22) Filed: Aug. 1, 2024

Related U.S. Application Data
(63) Continuation of application No. PCT/CN2024/081071, filed on Mar. 12, 2024.

(30) Foreign Application Priority Data
Mar. 11, 2024 (CN) 202410272514.6

(51) Int. Cl. G01N 5/04; G01N 33/00

(52) U.S. Cl. CPC G01N 5/04; G01N 33/0098

(57) ABSTRACT

The invention concerns a method for analyzing the leaf group composition of a cigarette sample based on tobacco thermal characteristics, which comprises the following steps: (1) Preparing a to-be-analyzed cigarette sample and single-grade tobacco samples; (2) Collecting thermal analysis spectra of the cigarette sample and the single-grade tobacco samples; (3) Using a thermal analysis mapping algorithm to obtain the tobacco leaf composition and proportion of the cigarette sample. The method of the invention can complete the composition analysis of an unknown cigarette in a few minutes, and can obtain a clear formula composition and proportion value that is objective, efficient, versatile, has good repeatability and high sensitivity, and has unique advantages in cigarette analysis in the tobacco industry.

### CROSS-REFERENCE TO RELATED APPLICATIONS

This application is a continuation of International Pat. Appl. No. PCT/CN2024/081071, filed on Mar. 12, 2024, which claims the benefit of Chinese Pat. Appl. No. 202410272514.6, filed on Mar. 11, 2024, both of which are incorporated herein by reference as if fully set forth herein.

### TECHNICAL FIELD

The invention belongs to the technical field of tobacco, in particular to a method for analyzing the leaf group composition of a cigarette sample based on the thermal characteristics of tobacco leaves.

### BACKGROUND

The quality and characteristics of cigarettes are mainly designed using the proportion of various tobacco leaves of different origins, varieties and grades. It is usually necessary to rely on formula experience and sensory evaluation, and manually select 10-20 kinds of tobacco leaves from hundreds of stock grades of tobacco raw materials to carry out the formula design (i.e., using different proportions of different tobacco leaf groups or varieties). As a result, the composition of the coiled-tobacco leaf group is extremely complicated, and it is difficult to analyze the composition of an unknown coiled-tobacco leaf group manually. It is of great significance for the analysis and leaf design of competing tobacco products to realize the composition analysis of coiled tobacco leaves using instrument detection, objective data and a scientific or technical approach.

Thermogravimetric analysis (TG/DTA) can provide stable reaction conditions under programmed temperature conditions, and is an ideal experimental tool for tobacco pyrolysis research. Derivative thermogravimetric methods, also called derivative thermogravimetry, is derived from thermogravimetric methods, and is a technique that records the first derivative of a TG curve with respect to temperature or time. The result of the technique is the derivative thermogravimetric curve, or DTG curve. The characteristics of DTG curves are: they can accurately reflect the initial reaction temperature, maximum reaction rate temperature and reaction termination temperature of each stage of mass loss; and the area of each peak on the DTG curve is proportional to the mass loss of the corresponding sample on the TG curve. When the TG curve is not obvious to some steps in the heating process, the DTG curve can be clearly distinguished. The main feature of thermogravimetric methods are that they are highly quantitative and can accurately measure the mass change and the rate of mass or temperature change. According to this feature, as long as the mass of a substance changes when it is heated, it can be studied by thermogravimetric methods.

At present, the composition analysis of coiled tobacco leaves mostly adopts the combination of tobacco chemical composition analysis, flue gas chemical composition analysis, sensory evaluation and other analytic techniques. The work intensity is high, the subjectivity is strong, and the conclusions are unclear and without strong reference.

### SUMMARY

The present invention intends to solve the above problems.

Since a DTG curve can effectively represent the quality information of cut tobacco/tobacco leaves, the consistency between the analysis of the composition or formula and the actual formula of tobacco leaf groups or varieties can be simulated and evaluated by measuring and/or comparing DTG curves. In order to improve the generality and versatility of tobacco leaf group analysis and the workload of analysts, the present invention concerns a DTG differential correlation model and a combination and/or optimization algorithm for analysis of DTG curves to characterize and/or identify the quality information of cigarette cut tobacco by analyzing thermogravimetric spectra, automatically searching for the tobacco leaf ratio of a tobacco leaf group formula (e.g., in a cigarette sample), and analyzing the composition of the tobacco leaf group using objective data. The composition and proportion of the leaf groups or varieties in the formula can be clearly obtained, which is of great significance to the analysis and design of leaf group formulas of competing cigarettes.

The invention concerns a method for analyzing a leaf group composition of one or more cigarette samples based on thermal characteristics of tobacco. The specific step is to analyze a competing cigarette (e.g., a to-be-analyzed cigarette) on the basis of existing cigarette data (e.g., thermogravimetric spectral data for individual tobacco leaf groups or varieties), and obtain the specific tobacco composition and formula ratio therefrom.

The technical scheme of the invention is as follows:

A method of analyzing a leaf group composition of a cigarette sample based on thermal characteristics of tobacco leaves comprises the following steps: (1) Preparing a to-be-analyzed cigarette sample and single-grade tobacco samples; (2) Collecting thermal analysis spectra (e.g., thermogravimetric spectral data) of the cigarette sample and the single-grade tobacco samples; and (3) mapping the thermal analysis spectra of the plurality of single-grade tobacco samples to the thermal analysis spectrum or spectra of the cigarette sample (e.g., using a thermal analysis mapping algorithm) to obtain a leaf composition (e.g., a tobacco leaf composition, including the identity and proportion of the tobacco leaves corresponding to the single-grade tobacco samples) of the analyzed cigarette sample. The proportion(s) obtained may be of the single-grade tobacco samples that, in combination, provide minimal (e.g., the smallest) difference(s) from the thermal analysis spectrum of the cigarette sample according to the thermal analysis mapping algorithm.

Preferably, step (1) includes only one cigarette sample, and the number of the selected single-grade tobacco leaf samples is not less than 50. Each of the cigarette sample and the single-grade tobacco leaf samples may be held in a constant temperature and humidity environment (for example, at a temperature of 20-25° C. [e.g., 22° C.±1° C.] and a relative humidity of 40-70% [e.g., 60%+2%]) for not less than 12, 24 or 48 hours. Generally, the cigarette sample is not flavored, the sample has an initial mass of not less than 5 g (e.g., prior to TG analysis), and the cigarette sample (and optionally the single-grade tobacco leaf samples) may be crushed using a crushing mesh having a mesh size of not less than 100 mesh prior to thermal analysis. Generally, no less than 50 typical single-grade tobacco leaf samples are selected, but other, similar numbers (e.g., 25 or more, 40 or more, 100 or more, etc.) are also suitable. The single-grade tobacco leaf samples should include leaves having different grades, different origins and different parts. The taste of the single-grade tobacco leaf samples may also be different. The single-grade tobacco samples should each have an initial mass of not less than 5 g (e.g., prior to TG analysis), and the single-grade tobacco leaf samples may also be crushed prior to thermal analysis using a crushing mesh having a mesh size of not less than 100 mesh (e.g., the same crushing mesh as the cigarette sample).

Preferably, step (2), collection of a thermal analysis spectrum, comprises weighing a (5.00±0.05) mg sample and heating it in a thermogravimetric crucible (e.g., an alumina, platinum or other TG crucible) from an initial temperature of 30-100° C. (e.g., 50° C.) at a heating rate of 5-20° C./min (e.g., 10° C./min) to a termination temperature of 850-900° C. The termination temperature may be held for 1-10 min (e.g., 5 min). Heating may be conducted in a TG instrument or analyzer with a chamber through which nitrogen gas flows (e.g., for protection and/or thermal reaction of the sample) at a flow rate of 20 mL/min. The resulting TG spectrum may include temperature (° C.) as the X-axis and mass change (%) of the sample as the Y-axis (e.g., of a graph), and the TG data are plotted on the graph, which may result in a TG curve. Alternatively, the X-axis and Y-axis parameters may be switched (i.e., mass change of the sample may be plotted along the X-axis, and temperature along the Y-axis). In other or further alternatives, the TG spectrum may substitute mass of the sample (in mg) for mass change, and may substitute time (in min) for temperature. Before sample analysis, the thermogravimetric analyzer is set and kept at 900° C. for 10 min to clear the impurities in the thermogravimetric crucible, and the empty crucible is used as a reference (e.g., a TG spectrum is generated using the empty crucible as a control). The sensitivity of the balance in the thermogravimetric analyzer/instrument may be not less than 0.1 μg, and the curve resolution (e.g., of the TG graph or spectrum) may be not less than 50 million resolution points.

Preferably, step (3) (e.g., to study and obtain the tobacco composition and proportion of the cigarette sample to be analyzed) includes the following sub-steps:
(A) obtaining a first derivative of the TG data/curve with respect to time or temperature to get a differential weight loss (DTG) curve, a DTG matrix (which may be represented by the letter Y) of the cigarette sample, and a DTG matrix (which may be represented by the letter X, where X=[x₁ x₂ . . . xₙ]) of each of the single-grade tobacco samples, where n is the number of the single-grade tobacco samples;
(B) Selection of temperature segment: selecting a segment of the DTG curves corresponding to a specific temperature segment within a temperature range of 50° C.-900° C. as actual analysis curves (e.g., to provide characteristic DTG matrices for each of the cigarette sample and the plurality of single-grade tobacco samples in which the first derivatives of the TG data/curves have the greatest variation);
(C) Determination of the objective function: determining (or selecting) an objective function for the purpose of optimization (e.g., for optimizing a combination of the characteristic DTG matrices of the plurality of single-grade tobacco samples to match the characteristic DTG matrix of the cigarette sample);
(D) Determination of restrictions: determining or setting restrictions according to one or more actual fitting operations, to ensure the accuracy of the calculation(s) and avoid wasting computing resources (e.g., determining or setting fitting restrictions for optimizing the combination of the characteristic DTG matrices of the single-grade tobacco samples to match [e.g., enable minimizing differences from] the characteristic DTG matrix of the cigarette sample); and
(E) Determination of an optimization method: optimizing the combination of the characteristic DTG matrices of the single-grade tobacco samples that has a minimal difference from the characteristic DTG matrix of the cigarette sample (e.g., using linear constraint conditions and an appropriate objective function) to obtain a single-grade tobacco composition coefficient and proportion (e.g., an identity and proportion of single-grade tobacco leaves corresponding to the single-grade tobacco samples) that are most similar to the cigarette sample as the tobacco leaf composition of the cigarette sample.

Preferably, the temperature segment in the sub-step (B) is 100-400° C. (e.g., 150-380° C., 100-200° C., etc.).

Preferably, the objective function in the sub-step (C) is one of the following:
(a) F=1/corr((c*X), Y), where corr is a correlation coefficient calculation, and c is a combination coefficient of single-grade tobacco leaves;
(b) F=sum (sqrt (((c*X−Y)·/Y).\hat{ }2)), where sqrt is a root-mean-square calculation, sum is a summation calculation, and c is the combination coefficient of single-grade tobacco leaves; or
(c) F=sqrt (sum ((c*X−Y).\hat{ }2)/sum (Y.\hat{ }2)), where sqrt is the root-mean-square calculation, sum is the summation calculation, and c is the combination coefficient of single-grade tobacco leaves.

Preferably, the restriction conditions in sub-step (D) may include one or more of:

$$1 = {\sum }_{i}^{n} c , \frac{c}{0.025} = z ,$$

c∈[0,1], and cᵢ>p . . . , where z is a natural number that is not 0 and p is a specific number, such as 0.01, 0.02 or 0.025. Similar to c (the combination coefficient of single-grade tobacco leaves) in the objective function, c; may be (i) a correlation coefficient of an individual single-grade tobacco leaf or (ii) a composition coefficient of an individual single-grade tobacco leaf in the composition of the cigarette sample. Thus, c; may be a positive integer multiple of p. Respectively, the sum of the composition coefficients (e.g., of the single-grade tobacco samples in the leaf composition) may be 1, the composition ratio of the leaf composition (e.g., the unit proportion of any particular single-grade tobacco sample in the leaf composition) may be an integer multiple of 0.01, 0.02, 0.025 or other multiple of 0.05 or 0.1 between 0 and 1 (e.g., >0 and ≤0.1), and the composition value of a certain cigarette (e.g., the individual proportions of particular single-grade tobacco leaves in the cigarette sample composition as determined by the present method) is not less than p (or p . . . ). In various examples, the fitting restrictions further include c/m=z, where m is selected from 0.01, 0.02 and 0.025, and z is an integer of at least 10 (e.g., at least 10, 20, 25, 40, 50, 100, etc.).

Preferably, the optimization method for sub-step (E) comprises one or more global optimization algorithms, gradient descent algorithms, and/or genetic algorithms. The global optimization, gradient descent, and genetic algorithms may be selected from known global optimization, gradient descent, and genetic algorithms.

In further aspects, the invention may concern a method of formulating a cut tobacco composition, comprising the present of analyzing a leaf group composition of a cigarette sample, then combining the single-grade tobacco leaves in the same or similar proportions as those found in the tobacco leaf composition obtained by the thermal analysis mapping algorithm to form or formulate the cut tobacco composition. Examples of cut tobacco compositions having proportions similar to those found in the tobacco leaf composition obtained by the thermal analysis mapping algorithm include variations of from 1% to 5% (i.e., 0.01-0.05) in from 1 to 5 (preferably 1-3) of the single-grade tobacco leaves, thereby enabling improvements in certain qualities of the cut tobacco composition relative to the composition of the analyzed cigarette sample, reductions in cost without a significant reduction in quality, etc. Typically, the number of different single-grade tobacco leaves in the cut tobacco composition is at least 10 (e.g., 10-50). Although the to-be-analyzed cigarette sample may comprise the same or different proportions of the same or different tobacco leaves as those in the single-grade tobacco samples, the concept is to match or surpass the qualities of the tobacco in the cigarette sample with the qualities of the cut tobacco composition (which is based on the obtained tobacco leaf composition).

The invention has the following beneficial effects:

1. The method of the invention provides thermal analysis spectra of finished cigarettes and single-grade tobacco leaves, and enables obtaining the composition and the proportion of single-grade tobacco leaves that are similar or identical to (e.g., having a sensory quality of) those of the to-be-analyzed cigarette by selecting an appropriate objective function, setting appropriate restriction conditions and determining one or more appropriate optimization methods through a dynamic study using a programmed thermogravimetric temperature increase. The method of the invention can complete the composition analysis of a finished (e.g., commercial) cigarette within a few minutes, and can greatly reduce the analyst's workload and the number of tests. It is objective, efficient, versatile, with good repeatability and high sensitivity, and has unique advantages in the analysis of finished cigarettes in the tobacco industry.

2. The method of the invention avoids wet chemical operations such as chemical composition analysis of cut tobacco (which may use a large amount of chemicals), chemical composition analysis of flue gas, and so on that may be required for the analysis of the conventional tobacco coiled leaf groups, and turns instead to a dry chemical operation, which is simple, uses a minimal sample (e.g., 10 mg or less), is non-toxic and harmless, and causes no harm to the operator and no environmental pollution.

3. The method of the invention provides specific formulas and cigarette composition design objectives, abundant data support and digital programmatic technology for the development and analysis of cigarette products, realizes automatic search and objective evaluation of cigarette product formula design schemes, and can avoid the traditional dependence on expert experience and sensory evaluation of subjective factors and differential characterization.

### EXAMPLES

The present invention is further explained by the embodiments below, but is not limited to the present embodiments. Experimental methods not specified in the embodiments are generally known or commercially available in accordance with conventional conditions, conditions described in a manual, or general equipment, materials, reagents, etc. used in accordance with conditions suggested by the manufacturer, unless otherwise specified. The raw materials in the following embodiments and the reference samples are commercially available.

Example: method of analyzing the composition and proportion of coiled tobacco leaf groups of a well-known domestic brand cigarette product sample (to be analyzed), using the following steps:

(1) Select a product sample of a well-known domestic brand of cigarettes (the to-be-analyzed cigarette) and 50 single-grade tobacco samples of different origin, different parts and different grades of 5 grams each (single-grade tobacco), and pass both the to-be-analyzed cigarette sample and single-grade tobacco leaf samples through a 100-mesh screen, then keep the to-be-analyzed cigarette sample and single-grade tobacco leaf samples in a constant temperature and humidity environment (22+1° C., relative humidity of 60+2%) for 48 hours.

(2) Before sample analysis, the thermogravimetric analyzer is kept at 900° C. for 10 min to clear the impurities in the TG crucible and furnace, and the empty thermogravimetric crucible is used for reference (e.g., as a control sample for a reference/blank TG spectrum). A 5.00 (+0.05) mg sample was weighed and placed in the thermogravimetric crucible (e.g., a platinum TG crucible). The temperature in the TG furnace was increased from an initial temperature of 50° C. or 30° C. to a termination temperature of 850° C. or 900° C. at a rate of 10° C./min, then the TG furnace temperature was held at the termination temperature for 5 min. Nitrogen gas (for both protection and reaction) flowed through the TG furnace at a flow rate of 20 mL/min. The TG test results (e.g., the decrease in mass of the sample in percent as a function of temperature) were plotted on a graph with temperature (C) as the X-axis and the mass change of the sample (%) as the Y-axis. The derived data were TG result data.

(3) The differential weight loss curve data (DTG matrices) can be obtained by taking the first derivative of the curve representing mass change with respect to time (derived from the TG test result graph) for each sample. Since the temperature increases linearly with time, one can substitute time for temperature on the TG curves and get substantially the same results. The DTG matrix Y for the analyzed cigarette sample and the DTG matrix X=[x1 x2 . . . x50] for single-grade tobacco leaves are shown in Table 1 and Table 2 below.

TABLE 1-1
Cigarette DTG matrix Y

| | Temperature ° C. | | | | | | |
|---|---|---|---|---|---|---|---|
| | 30 | 31 | . . . | 45 | 46 | . . . | 900 |
| Cigarette | −2.62E−05 | −2.62E−05 | . . . | −2.81E−05 | −3.62E−05 | . . . | −5.43E−05 |
| sample | | | | | | | |

TABLE 2-1
Single grade tobacco DTG matrix X

| | Temperature ° C. | | | | | | |
|---|---|---|---|---|---|---|---|
| | 30 | 31 | . . . | 45 | 46 | . . . | 900 |
| Tobacco leaf | −3.86E−05 | −3.86E−05 | . . . | −4.07E−05 | −4.94E−05 | . . . | −4.51E−05 |
| 1 | | | | | | | |
| Tobacco leaf | −2.53E−05 | −2.53E−05 | . . . | −2.73E−05 | −3.54E−05 | . . . | −5.27E−05 |
| 2 | | | | | | | |
| . . . | . . . | . . . | . . . | . . . | . . . | . . . | . . . |
| Tobacco leaf | −2.77E−05 | −2.77E−05 | . . . | −2.96E−05 | −3.78E−05 | . . . | −4.72E−05 |
| 50 | | | | | | | |

(4) Select the DTG data corresponding to the temperature range 100-200° C. as the specific temperature segment (e.g., X_New and Y_New):

TABLE 1-2
Cigarette DTG matrix Y_New

| | Temperature ° C. | | | | | | |
|---|---|---|---|---|---|---|---|
| | 100 | 101 | . . . | 180 | 181 | . . . | 200 |
| Cigarette | −3.94E−05 | −3.94E−05 | . . . | −3.71E−05 | −3.92E−05 | . . . | −4.13E−05 |
| sample | | | | | | | |

TABLE 2-2
Single-grade tobacco DTG matrix X_New

| | Temperature ° C. | | | | | | |
|---|---|---|---|---|---|---|---|
| | 100 | 101 | . . . | 180 | 181 | . . . | 200 |
| Tobacco leaf | −3.16E−05 | −3.36E−05 | . . . | −3.57E−05 | −3.94E−05 | . . . | −4.11E−05 |
| 1 | | | | | | | |
| Tobacco leaf | −3.22E−05 | −2.46E−05 | . . . | −3.73E−05 | −3.94E−05 | . . . | −4.26E−05 |
| 2 | | | | | | | |
| . . . | . . . | . . . | . . . | . . . | . . . | . . . | . . . |
| Tobacco leaf | −2.67E−05 | −2.98E−05 | . . . | −3.66E−05 | −3.88E−05 | . . . | −4.32E−05 |
| 50 | | | | | | | |

(5) The objective function is set to F=1/corr((c*X),Y), the limited parameter is 1=Σᵢⁿc, and the parameter calculation based on a known global optimization algorithm is established. For example, Python code for the known global optimization algorithm may include:
from scipy import stats
import numpy as np

Exemplary code for the restriction conditions (e.g., based on correlation coefficients and/or a fitting operation) may include:
def fitness (c):
   return 1/stats.pearsonr (np.dot(Data_New, c), Target_New).statistic Qualification condition
m,n=Data_New.shape
x0=np.random.random(n)#0-1 [A random number between 0 and 1, optionally a multiple of a minimum value such as 0.01 or 0.025, is the initial value]
bnds=[(0, 1)]*n #[Define the coefficient value interval]
cons=({‘type’: ‘eq’, ‘fun’: lambda x: sum (x)−1}) #[The coefficients add up to 1]

The above text in brackets may not be code, but may explain the function or meaning of the code.

Exemplary code for a global search and/or optimization algorithm such as basinhopping that finds a global minimum (e.g., differences between the results of the objective function for various combinations of the single-grade tobacco leaves according to the restriction conditions relative to the DTG data in the specific temperature segment or selected temperature range) may include:
from scipy.optimize import basinhopping
minimizer_kwargs={“method”: “SLSQP”,
   “constraints”: cons,
   “bounds”: bnds,}
ret=basinhopping (fitness, x0, minimizer_kwargs-minimizer_kwargs, niter-200)

The results of the calculation(s) according to the objective function, the restrictions (e.g., for fitting purposes), and the basinhopping global search/optimization as described above are shown in Table 3 below:

TABLE 3
Corresponding leaf formulations for calculation

| Single grade | Formula ratio/ |
|---|---|
| tobacco leaf | composition value |
| Tobacco leaf 2 | 0.05 |
| Tobacco leaf 3 | 0.12 |
| Tobacco leaf 4 | 0.10 |
| Tobacco leaf 14 | 0.05 |
| Tobacco leaf 15 | 0.08 |
| Tobacco leaf 20 | 0.05 |
| Tobacco leaf 25 | 0.10 |
| Tobacco leaf 26 | 0.05 |
| Tobacco leaf 27 | 0.05 |
| Tobacco leaf 32 | 0.12 |
| Tobacco leaf 35 | 0.05 |
| Tobacco leaf 42 | 0.05 |
| Tobacco leaf 46 | 0.05 |
| Tobacco leaf 50 | 0.08 |

Verification experiment: According to the proportions/formula ratio of the 14 single-grade tobacco leaves shown in Table 3 above, a sample of cigarette cut tobacco having the formula of Table 3 was prepared. A panel of nine sensory evaluation experts evaluated and scored the differences in the sensory qualities of (1) the cigarette cut tobacco sample having the formula of Table 3 and (2) the sample of the well-known domestic brand of cigarette that was analyzed in the above example. The results showed no differences in the sensory evaluation.

The above embodiments only disclosed several embodiments of the invention, and their descriptions are more specific and detailed, but they cannot be construed as limitations on the scope of the invention. It should be noted that for ordinary technicians in the field, without deviating from the concepts of the invention, a number of derivations and improvements can be made that are within the scope of protection of the invention. Therefore, the scope of protection of the invention patent shall be subject to the attached claims.

What is claimed is:

1. A method for analyzing a cigarette sample, comprising: (1) preparing the cigarette sample and a plurality of single-grade tobacco samples; (2) collecting thermal analysis spectral data of the cigarette sample and each of the plurality of single-grade tobacco samples; and (3) mapping the thermal analysis spectral data of the plurality of single-grade tobacco samples to the thermal analysis spectral data of the cigarette sample to obtain a leaf composition of the cigarette sample, wherein mapping the thermal analysis spectra of the plurality of single-grade tobacco samples to the thermal analysis spectrum of the cigarette sample comprises:
(A) obtaining a first derivative of thermogravimetric (TG) spectral curves with respect to time or temperature for each of the cigarette sample and the plurality of single-grade tobacco samples to get a differential weight loss (DTG) curve and a DTG matrix for each of the cigarette sample and the single-grade tobacco samples;
(B) selecting a segment of the DTG curves corresponding to a specific temperature segment within the temperature range of 50° C.-900° C. to provide characteristic DTG matrices for each of the cigarette sample and the plurality of single-grade tobacco samples;
(C) determining an objective function for optimizing a combination of the characteristic DTG matrices of the plurality of single-grade tobacco samples to match the characteristic DTG matrix of the cigarette sample;
(D) determining or setting fitting restrictions for optimizing the combination of the characteristic DTG matrices of the plurality of single-grade tobacco samples to match the characteristic DTG matrix of the cigarette sample; and
(E) optimizing the combination of the characteristic DTG matrices of the plurality of single-grade tobacco samples that has a minimal difference from the characteristic DTG matrix of the cigarette sample to obtain an identity and proportion of single-grade tobacco leaves that are most similar to the cigarette sample as the leaf composition of the cigarette sample.

2. The method of claim 1, wherein the specific temperature segment is 100-400° C.

3. The method of claim 1, wherein the objective function is selected from:
(a) F=1/corr((c*X), Y), where F is the objective function, corr is a correlation coefficient calculation, and c is a combination coefficient of the single-grade tobacco samples;
(b) F=sum(sqrt(((c*X−Y)·/Y)·\hat{ }2)), where sqrt is a root-mean-square calculation, sum is a summation calculation, and c is the combination coefficient of single-grade tobacco samples; and
(c) F=sqrt (sum((c*X−Y)·\hat{ }2)/sum(Y·\hat{ }2)), where sqrt is the root-mean-square calculation, sum is the summation calculation, and c is the combination coefficient of single-grade tobacco samples.

4. The method of claim 3, wherein the objective function is F=1/corr((c*X),Y).

5. The method of claim 3, wherein the objective function is F=sum (sqrt (((c*X−Y)·/Y)·\hat{ }2)) or F=sqrt (sum((c*X−Y)·\hat{ }2)/sum(Y·\hat{ }2)).

6. The method of claim 1, wherein the fitting restrictions include 1=Σᵢⁿc, where c is a combination coefficient of the single-grade tobacco samples, and cᵢ≥0.01.

7. The method of claim 6, wherein the fitting restrictions further include c/m=z, where m is selected from 0.01, 0.02 and 0.025, and z is a non-zero natural number.

8. The method of claim 7, wherein m is 0.025 and z is an integer of at least 10.

9. The method of claim 6, wherein the fitting restrictions further include c∈[0,1].

10. The method of claim 6, wherein the fitting restrictions further include cᵢ>p, where p is a specific number.

11. The method of claim 10, wherein p is selected from 0.01, 0.02 and 0.025, and cᵢ is a positive integer multiple of p.

12. The method of claim 1, wherein the combination of the characteristic DTG matrices of the plurality of single-grade tobacco samples are optimized using one or more global optimization algorithms, gradient descent algorithms, or genetic algorithms.

13. The method of claim 1, wherein the single-grade tobacco leaves correspond to the single-grade tobacco samples.

14. A method of formulating a cut tobacco composition, comprising:
the method of claim 1, then
combining the single-grade tobacco leaves in proportions identical or similar to those in the leaf composition to form or formulate the cut tobacco composition.

15. The method of claim 14, comprising combining the single-grade tobacco leaves in proportions similar to those in the leaf composition.

16. The method of claim 15, wherein the cut tobacco composition having proportions similar to those found in the leaf composition includes a variation of from 0.01 to 0.05 in the proportion(s) of from 1 to 5 of the single-grade tobacco leaves in the leaf composition.

17. The method of claim 16, wherein the variation is from 0.02 to 0.05 in the proportion(s) of from 1 to 3 of the single-grade tobacco leaves in the leaf composition.
